(19) United States
(12) Patent Application Publication
(10) Pub. No.: US 2007/0191957 A1
(43) Pub. Date: Aug. 16, 2007

(54) SPINAL IMPLANTS WITH COOPERATING SUTURE ANCHORS

(75) Inventors: Paul Anderson, Verona, WI (US); Guilhem Denoziere, Atlanta, GA (US); John W. McClellan III, Omaha, NE (US); Edward Miller, Cincinnati, OH (US)

Correspondence Address: MYERS BIGEL SIBLEY & SAJOVEC, PO BOX 37428, RALEIGH, NC, 27627, US

(73) Assignee: SpineMedica Corporation

(21) Appl. No.: 11/671,507

(22) Filed: Feb. 6, 2007

Related U.S. Application Data
(60) Provisional application No. 60/765,984, filed on Feb. 7, 2006.

(51) Int. Cl. A61F 2/44; A61F 2/28

(52) U.S. Cl. 623/17.16; 623/23.52

(57) ABSTRACT

Spinal implants have cooperating suture anchors. The devices include: (a) a spinal implant; and (b) at least one suture anchor comprising a threaded bone anchor holding at least one suture extending outwardly therefrom. In position, the at least one suture extends outward from the threaded bone anchor and attaches to the spinal implant while the threaded anchor is anchored in a vertebral body.

### RELATED APPLICATIONS

This application claims priority to U.S. Provisional Application Ser. No. 60/765,984, filed Feb. 7, 2006, the content of which is hereby incorporated herein by reference as if recited in full herein.

### FIELD OF THE INVENTION

The invention relates to spinal implants.

### BACKGROUND OF THE INVENTION

The vertebrate spine is made of bony structures called vertebral bodies that are separated by relatively soft tissue structures called intervertebral discs. The intervertebral disc is commonly referred to as a spinal disc. The spinal disc primarily serves as a mechanical cushion between the vertebral bones, permitting controlled motions between vertebral segments of the axial skeleton. The disc acts as a joint and allows physiologic degrees of flexion, extension, lateral bending, and axial rotation. The disc must have sufficient flexibility to allow these motions and have sufficient mechanical properties to resist the external forces and torsional moments caused by the vertebral bones.

The normal disc is a mixed avascular structure having two vertebral end plates (“end plates”), an annulus fibrosis (“annulus”) and a nucleus pulposus (“nucleus”). Typically, about 30-50% of the cross sectional area of the disc corresponds to the nucleus. Generally described, the end plates are composed of thin cartilage overlying a thin layer of hard, cortical bone that attaches to the spongy cancellous bone of the vertebral body. The end plates act to attach adjacent vertebrae to the disc.

The annulus of the disc is a relatively tough, outer fibrous ring. For certain discs, particularly for discs at lower lumbar levels, the annulus can be about 10 to 15 millimeters in height and about 10 to 15 millimeters in thickness, recognizing that cervical discs are smaller.

Inside the annulus is a gel-like nucleus with high water content. The nucleus acts as a liquid to equalize pressures within the annulus, transmitting the compressive force on the disc into tensile force on the fibers of the annulus. Together, the annulus and nucleus support the spine by flexing with forces produced by the adjacent vertebral bodies during bending, lifting, etc.

The compressive load on the disc changes with posture. When the human body is supine, the compressive load on the third lumbar disc can be, for example, about 200 Newtons (N), which can rise rather dramatically (for example, to about 800 N) when an upright stance is assumed. The noted load values may vary in different medical references, typically by about ±100 to 200 N. The compressive load may increase, yet again, for example, to about 1200 N, when the body is bent forward by only 20 degrees.

The spinal disc may be displaced or damaged due to trauma or a degenerative process. A disc herniation occurs when the annulus fibers are weakened or torn and the inner material of the nucleus becomes permanently bulged, distended, or extruded out of its normal, internal annular confines. The mass of a herniated or “slipped” nucleus tissue can compress a spinal nerve, resulting in leg pain, loss of muscle strength and control, and even paralysis. Alternatively, with discal degeneration, the nucleus loses its water binding ability and deflates with subsequent loss in disc height. Subsequently, the volume of the nucleus decreases, causing the annulus to buckle in areas where the laminated plies are loosely bonded. As these overlapping plies of the annulus buckle and separate, either circumferential or radial annular tears may occur, potentially resulting in persistent and disabling back pain. Adjacent, ancillary facet joints will also be forced into an overriding position, which may cause additional back pain. The most frequent site of occurrence of a herniated disc is in the lower lumbar region. The cervical spinal disks are also commonly affected.

There are several types of treatment currently being used for treating herniated or degenerated discs: conservative care, discectomy, nucleus replacement, fusion and prosthesis total disc replacement (TDR). It is believed that many patients with lower back pain will get better with conservative treatment of bed rest. For others, more aggressive treatments may be desirable.

Discectomy can provide good short-term results. However, a discectomy is typically not desirable from a long-term biomechanical point of view. Whenever the disc is herniated or removed by surgery, the disc space will narrow and may lose much of its normal stability. The disc height loss may cause osteo-arthritis changes in the facet joints and/or compression of nerve roots over time. The normal flexibility of the joint is lost, creating higher stresses in adjacent discs. At times, it may be necessary to restore normal disc height after the damaged disc has collapsed.

Fusion is a treatment by which two vertebral bodies are fixed to each other by a scaffold. The scaffold may be a rigid piece of metal, often including screws and plates, or allo or auto grafts. Current treatment is to maintain disc space by placement of rigid metal devices and bone chips that fuse two vertebral bodies. The devices are similar to mending plates with screws to fix one vertebral body to another one. Alternatively, hollow metal cylinders filled with bone chips can be placed in the intervertebral space to fuse the vertebral bodies together (e.g., LT-Cage™ from Sofamor-Danek or Lumbar I/F CAGE™ from DePuy). These devices have disadvantages to the patient in that the bones are fused into a rigid mass with limited, if any, flexible motion or shock absorption that would normally occur with a natural spinal disc. Fusion may generally eliminate symptoms of pain and stabilize the joint. However, because the fused segment is fixed, the range of motion and forces on the adjoining vertebral discs can be increased, possibly enhancing their degenerative processes.

Some recent TDR devices have attempted to allow for motion between the vertebral bodies through articulating implants that allow some relative slippage between parts (e.g., ProDisc®, Charite™). See, e.g., U.S. Pat. Nos. 5,314,477, 4,759,766, 5,401,269 and 5,556,431. As an alternative to the metallic-plate, multi-component TDR (total disc replacement) designs, a flexible solid elastomeric spinal disc implant that is configured to simulate natural disc action (i.e., can provide shock absorption and elastic tensile and compressive deformation) is described in U.S. Patent Application Publication No. 2005/0055099 to Ku, the contents of which are hereby incorporated by reference as if recited in full herein.

Other parts of the spine may also deteriorate and/or need repair and implants for various portions of the spine may be desirable.

### SUMMARY OF EMBODIMENTS OF THE INVENTION

Embodiments of the present invention are directed to anchoring spinal implants in bone using suture anchors.

Some embodiments are directed to spinal implants with cooperating suture anchors. The devices include a spinal implant and at least one suture anchor comprising a threaded bone anchor holding at least one suture. In position, the at least one suture extends outwardly from the threaded bone anchor and attaches to the spinal implant while the threaded bone anchor is anchored in a vertebral body.

Other embodiments are directed to medical spinal implant kits. The kits include; (a) a total disc replacement (TDR) spinal implant comprising a bone attachment material; and (b) a plurality of suture anchors configured to define suture knots against an outer surface of the bone attachment material with the threaded anchors configured and sized to reside in at least one vertebral body above or below the TDR implant to secure the TDR implant in position.

Still other embodiments are directed to methods of attaching a total disc replacement (TDR) implant to at least one vertebral body. The methods include: (a) implanting a TDR; (b) anchoring at least one bone anchor in at least one vertebral body proximate the TDR; and (c) tying at least one suture set attached to the bone anchor to the TDR to thereby secure the TDR in position in the body.

Some embodiments are directed to TDR implants. The implants include: (a) a flexible implant body; and (b) a bone attachment member with at least one outwardly extending plug configured and sized to reside in a cavity formed in a vertebral body.

The TDR implant may optionally include at least one threaded bone anchor with at least one suture set attached to the bone attachment member. A single anchor can be sized and configured to reside in the vertebral cavity with a respective plug.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is an anterior view of an implantable spinal disc prosthesis with cooperating suture anchors according to embodiments of the present invention,

FIG. 2A is an anterior view of another implantable spinal disc prosthesis with cooperating suture anchors according to embodiments of the present invention.

FIG. 2B is an anterior view of another implantable spinal disc prosthesis with cooperating suture anchors according to embodiments of the present invention.

FIG. 3 is an anterior view of a vertebral body with exemplary locations for suture anchors according to embodiments of the present invention.

FIGS. 4A and 4B are lateral views of a portion of a suture anchor held in vertebral bone according to embodiments of the present invention.

FIG. 5 is a side view of an exemplary suture anchor with a plurality of suture sets according to some embodiments of the present invention.

FIG. 6 is an exploded anterior view of a suture anchor with two suture sets and an implant according to embodiments of the present invention.

FIG. 7A-7E are sequential views of implantation steps that can be used to anchor a spinal implant according to embodiments of the present invention. FIGS. 7A-7C and 7E are lateral views and FIG. 7D is an anterior exploded view.

FIG. 8 is an anterior view of implantable spinal discs using several exemplary different suture anchor configurations according to embodiments of the present invention.

FIG. 9 is a schematic illustration of a medical kit according to embodiments of the present invention.

FIG. 10A is a lateral view of a bone attachment material comprising a plug configuration according to embodiments of the present invention.

FIG. 10B is a side perspective view of an exemplary bone cavity plug according to embodiments of the invention.

FIG. 11A is a lateral view of a spinal implant with bone attachment material comprising plugs or inserts according to embodiments of the present invention.

FIG. 11B is an anterior view of the device shown in FIG. 11A.

FIG. 12 is a side perspective view of a spinal implant with keels according to some embodiments of the present invention.

FIG. 13A is a side view of a portion of the spine illustrating an implant on a spinous process with a cooperating suture anchor according to embodiments of the present invention.

FIG. 13B is a side view of an exemplary spinous process cuff suitable for use with cooperating suture anchors according to some embodiments of the present invention.

FIG. 14 is a side view of a spine illustrating a wide range facet prosthesis secured using a cooperating suture anchor according to some embodiments of the present invention.

### DETAILED DESCRIPTION

The present invention now is described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. The sequence of operations (or steps) is not limited to the order presented in the claims or figures unless specifically indicated otherwise.

The terms “spinal disc implant” and “spinal disc prosthesis” are used interchangeably herein to designate total disc replacements using an implantable total disc replacement (TDR) prosthesis (rather than a nucleus only) and as such are configured to replace the natural spinal disc of a mammalian subject (for veterinary or medical (human) applications). In contrast, the term “spinal implant” refers to both TDR spinal disc implants and alternative spinal implants, such as, for example, a spinal annulus implant, a spinal nucleus implant, a facet implant, and a spinous process implant as well as implants for other portions of the spine.

The term “keel” means an implant component, feature or member that is configured to be received in a recess or mortise in an adjacent bone to facilitate short and/or long-term fixation and/or to provide twist or torsion resistance in situ.

The term “flexible” means that the member can be flexed or bent. In some embodiments, the implant can include a keel, which may be flexible but has sufficient rigidity to be substantially self-supporting so as to be able to substantially maintain a desired configuration outside of the body. If flexible, the keel can include reinforcement to increase its rigidity.

The term “mesh” means any flexible material in any form including, for example, knotted, braided, extruded, stamped, knitted, woven or otherwise, and may include a material with a substantially regular foramination pattern and/or irregular foramination patterns.

The term “macropores” refers to apertures having at least about a 1 mm diameter or width size, typically a diameter or width that is between about 1 mm to about 3 mm, and more typically a diameter or width that is between about 1 mm to about 1.5 mm (the width dimension referring to non-circular apertures). Where mesh keels are used, the macropores are larger than the openings or foramina of the mesh substrate. The macropores may promote bony through-growth for increased fixation and/or stabilization over time.

The term “loop” refers to a shape in the affected material that has a closed or nearly closed turn or figure. For example, the loop can have its uppermost portion merge into two contacting lower portions or into two proximately spaced apart lower portions. The term “fold” means to bend and the bend of the fold may have a sharp or rounded edge. The terms “pleat” or “fold” refer to doubling material on itself (with or without sharp edges). The term “attachment point” and derivatives thereof refers to a common attachment location and is not meant to restrict the attachment to a geometric point.

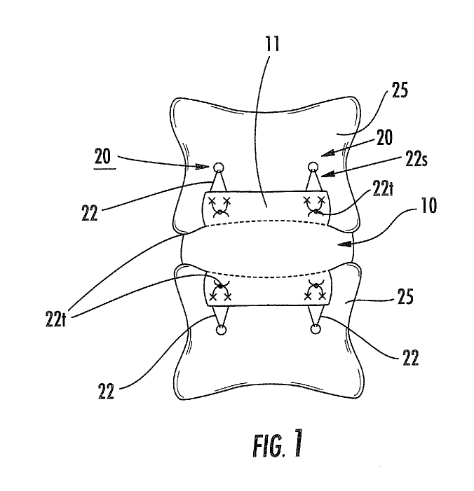

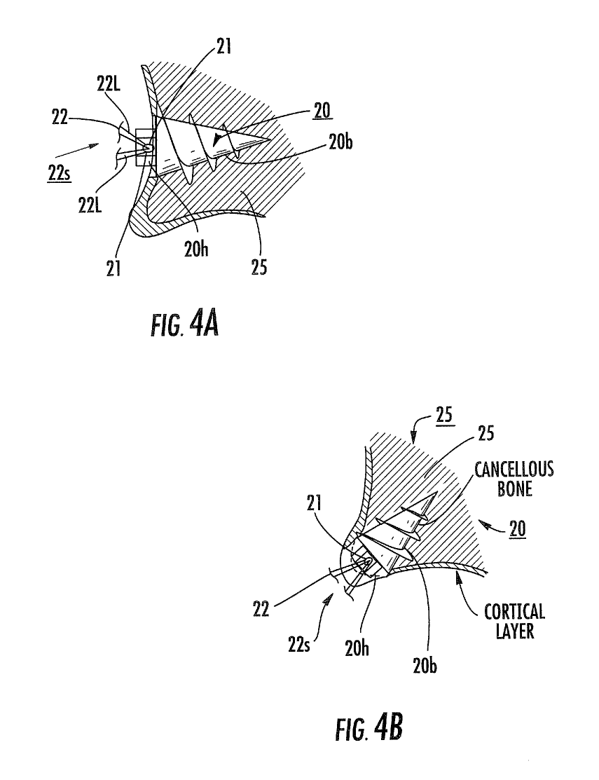

Referring now to the figures, FIG. 1 illustrates an example of a spinal implant 10 with cooperating suture anchors 20. The suture anchors 20 include at least one suture 22 that is attached to a bone anchor 20b (FIGS. 4A, 4B). Typically, the suture 22 is provided as a suture set 22s, in which each leg of the set is tied together such as using a knot 22t to secure the spinal implant 10 in location. The knot 22t can reside proximate to and/or against the outer surface of the implant 10. It is also noted that in lieu of, or with, the knot 22t, the ends of the sutures 22 may be attached to the implant 10 via other attachment means. For example, the two end portions of the suture 22 can be separately or jointly adhesively attached to the implant 10 such with an adhesive, heat-melt process, staple, clip or other anchor member.

The implant 10 can include a bone attachment member or material 11 that receives the suture 22. As shown, the bone attachment material 11 can reside above and below the primary body of the implant 10. However, the bone attachment material 11 may be configured to reside only above, only below, or to be substantially coextensive with the primary implant body (not shown). Each suture set 22s can be closed so that the respective knot 22t resides against or proximate an exterior surface of the bone attachment material 11, above or below the primary body of the implant 10. In some embodiments a unitary layer of bone attachment material can form a skirt that defines both an upper and lower bone attachment material 11. The bone attachment material 11 can comprise any biocompatible material suitable to provide the attachment and/or stabilization. The bone attachment material 11 may comprise a flexible substrate. In some embodiments, the bone attachment material 11 comprises a mesh substrate. The mesh can be metallic, fabric, polymeric or comprise combinations of materials.

The bone attachment material 11 can include one or more relatively small preformed apertures (not shown) at the respective target indicia markings 122 that can be sized and configured to receive the needle 23 and suture 22. The preformed apertures may be molded in or introduced at a manufacturing site to reduce clinician preparation time. Alternatively, the substrate can be configured to allow the needle to be inserted through the substrate in the target attachment regions in situ without using preformed apertures.

The bone attachment material 11 is typically between about 0.25 mm to about 20 mm thick, and is more typically between about 0.5 mm to about 5 mm thick. In some embodiments, the mesh comprises a DACRON mesh of about 0.7 mm thick available as Fablok Mills Mesh #9464 from Fablok Mills, Inc., located in Murray Hill, N.J. The mesh may comprise cryogel material to increase rigidity.

FIG. 1 illustrates that the implant 10 is secured using a plurality of suture anchors 20, some above and some below the implant 10. Although shown as four suture anchors 20, additional or lesser numbers of the suture anchors 20 may be used. Further, although the suture anchors 20 are shown as being substantially aligned (side to side and vertically) in proximate vertebral bone and in the bone attachment material 11, the suture anchors 20 may be arranged asymmetrically. In addition, bone screws or other devices may be used with one or more of the suture anchors 20 (not shown). The implant 10 can be attached to bone 25 using the cooperating suture anchors 20 in a manner that allows substantially normal, or at least not unduly restrictive, spinal movement.

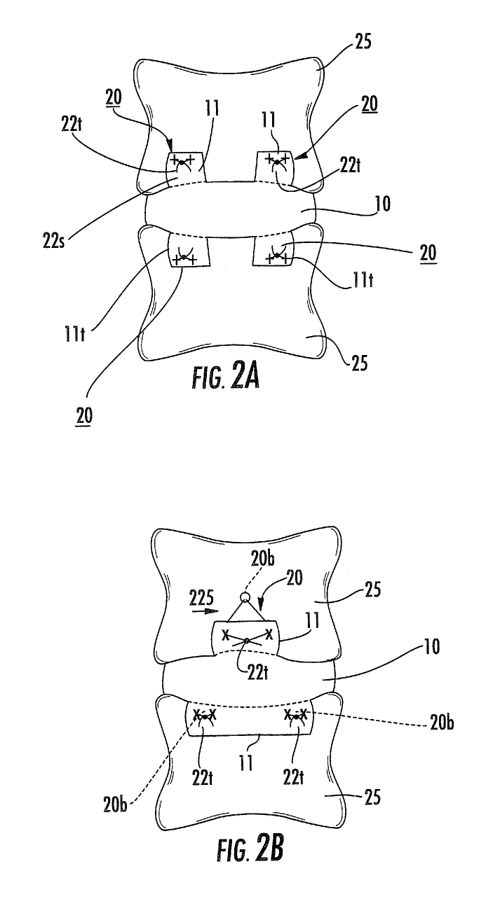

FIG. 2A illustrates that the bone attachment material 11 can be configured with discrete tabs lit spaced apart laterally; each tab lit can engage at least one suture set 22s. FIG. 2A also illustrates that the bone anchor 20b can reside under (behind) the bone attachment material 11, rather than above or below as shown in FIG. 1. FIG. 2B illustrates that the upper bone attachment material 11 may be configured differently from the lower bone attachment material. FIG. 2B also illustrates that the bone anchor 20b (FIG. 4A) may reside above the bone attachment material 11 while the lower bone anchors 20b (FIG. 4A) may reside substantially behind the bone attachment material 11. The mounting configuration can also be reversed with the lower bone screws 20b below the material 11 and the upper bone anchor 20b behind the material 11.

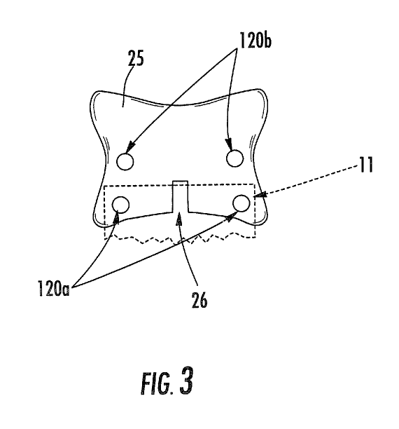

FIG. 3 illustrates a vertebral bone 25 with a mortise or keel recess 26 formed therein. The mortise or recess 26 can be formed into the vertebral bone 25 to accept fins or keels of implants (shown for example as feature 50 in FIG. 12). FIG. 3 illustrates exemplary bore locations 120a, 120b that can be used and/or formed by bone anchors 20b relative to the mortise 26. An implant 10 may employ bone anchors 20b at one or more of the bore locations. The bore locations 120a typically reside behind the bone attachment material 11 (shown in broken line) while the bore locations 120b typically reside above (or below) the material 11.

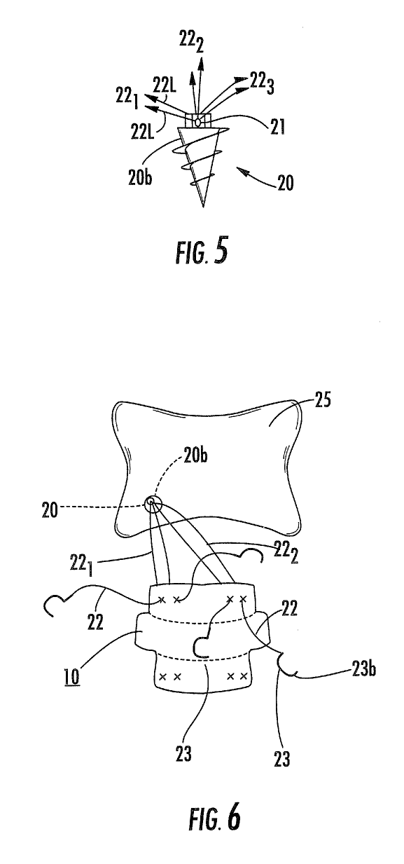

FIG. 4A illustrates a threaded bone anchor 20b with an attached suture set 22s in position in a vertebral body 25. As shown, a suture 22 is held by a head portion 20h of the bone anchor 20b. The head 20h can be recessed into or be substantially flush with the natural boundary of the vertebral bone 25. For recessed configurations, bone chips or other void filling (bone growth) material 325 (FIG. 9) may be inserted in the cavity between the material 11 and the bone anchor head 20h. The material 325 can be provided as part of a medical kit 500 (FIG. 9). Typically, the head 20h includes an aperture 21 and a length of suture 22 is threaded through the aperture 21 to form a suture set 22s with a pair of legs 22L. The opposing end portions of the suture legs 22L (the end portion away from the head 21) can include/merge into a needle 23 (FIG. 6). In use, after inserting the needle 23 through the bone attachment material 11, the corresponding suture leg 22L can be pulled through the material 11 and the suture set 22s can be tied or stitched together proximate an outside surface of the material 11.

FIG. 4B illustrates that the bone anchor 20b can be inserted through the cortical layer such that at least a tip portion thereof resides in cancellous bone. It is contemplated that the bone anchor 20b may have improved pullout strength if the threads of the bone anchor 20b bear on cortical bone. As shown, the bone anchor 20b can angularly reside in the bone 25 (rather than be substantially horizontal as shown in FIG. 4A). Combinations of these and other orientations may also be used.

FIG. 5 illustrates that the bone anchor 20b may be configured to hold a plurality of suture sets 22₁, 22₂, 22₃. Although shown as holding three, one or more of the bone anchors 20b may hold lesser or greater numbers of suture sets 22s. Each suture set 22₁, 22₂, 22₃ may be formed so that the respective sutures legs 22L have a different color or pattern for matching to allow easier alignment and/or attachment in situ. A template 300 (FIG. 9) may also be provided to help a clinician mark locations on vertebral bodies for the bone anchor 20b to help provide proper seating and alignment. The bone attachment material 11 may also include needle insertion indicia 122 (FIG. 9) to provide visual references that a clinician can use to attach the suture 22 to the implant 10. The indicia 122 may also be color coded to the suture for that location.

Also, although not shown, the bone anchor 20b may include a single suture leg rather than a suture set 22s. A first end portion can be integrally attached to the head of the bone anchor 20h with the other end portion including the needle 23. To attach to the bone attachment material 11, the single suture leg can be tied to another single leg or suture set or a discrete anchor member can be attached after the needle 23 is pulled through the material 11, or the single leg can be adhesively attached, stapled and/or clipped to the outer surface of the bone attachment material 11 (not shown).

The bone anchor 20b can be self-tapping and/or self-drilling. The bone anchor 20b may be implanted into a prior formed bore. The threads of the bone anchor 20b can be adapted to the porosity of the vertebral cancellous bone (which may be less dense than in other regions). The bone anchor 20b may have a largest diameter of between about 3-10 mm, typically between about 5-8 mm. The bone anchor 20b may have a length between about 8-30 mm, typically between about 10-20 mm.

FIG. 9 illustrates an alternate configuration of a bone anchor 20b. In this embodiment, the suture attachment region (aperture) 21 is recessed into the head 20h so that the threads extend substantially the entire length of the bone anchor body 20b. The threads can bear on the cortical layer of the vertebral body while still being substantially flush or slightly recessed with the outer layer of the vertebral body. This configuration may increase pull-out strength.

FIG. 6 illustrates that a first suture set 22₁ may be provided in a different length than a second suture set 22₂. Also, although shown as being attached to different corner portions of the bone material, the two suture sets 22₁, 22₂ may be attached adjacent each other in a common corner (side by side or one above the other) or one can be attached at a corner and the other at a medial portion. Other configurations may also be used.

The suture 22 and/or the bone anchor 20b may comprise a resorbable or non-resorbable biocompatible material.

As shown in FIG. 6, the needle 23 may be swaged, threaded or otherwise attached to the suture 22. The needle 23 may be straight or curved. As shown, the needle 23 is curved and may also include a substantially blunt tip 23b. Where a mesh is used to form the material 11, the blunt tip 23b may inhibit damage to mesh or other sensitive or susceptible fibers when suturing mesh material 11 to the bone. The suture legs 22L can have lengths between about 5-20 cm with the needles 23 on one end and the aperture or loop 21 of the head 20h at the other. The needle 23 is typically removed from the suture leg 22L after pulling the suture leg through the bone attachment material 11, and the suture leg 22L can be tied or otherwise secured to the material 11 and the surplus lengths thereof can be removed (cut).

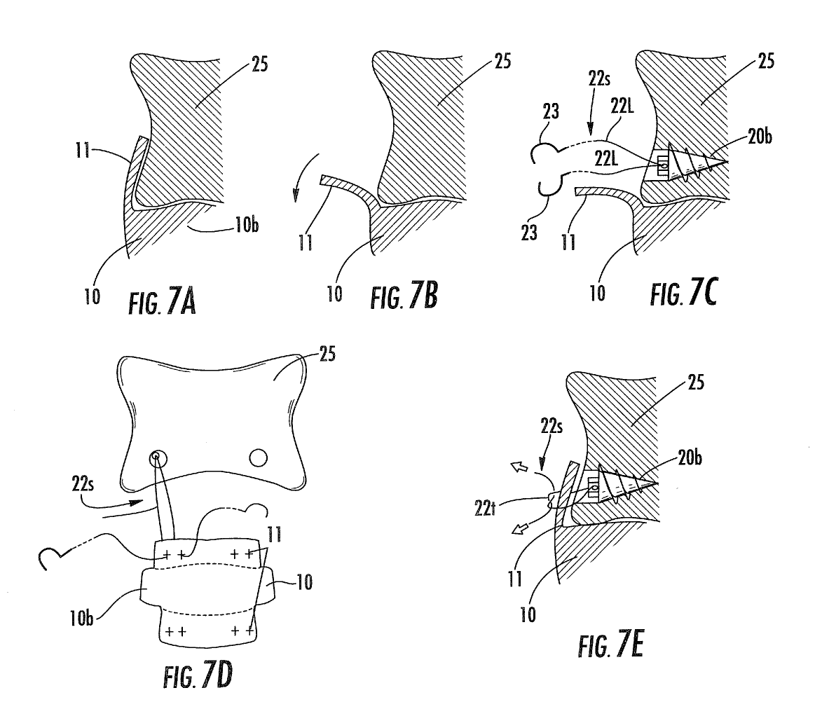

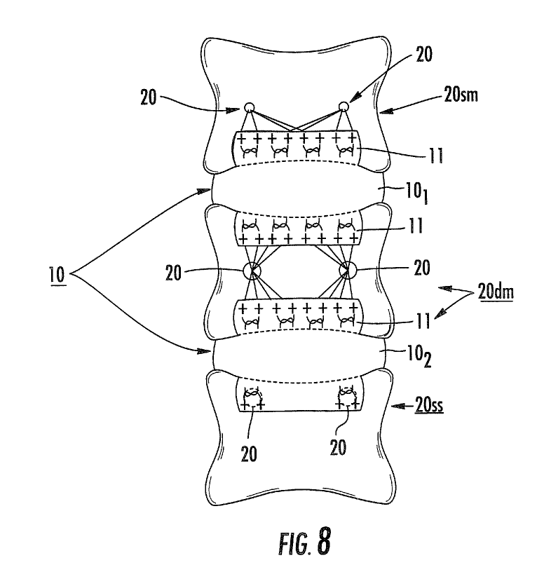

FIGS. 7A-7E illustrate a sequence of steps that can be used to attach a spinal implant to cooperating suture anchors 20 in situ. As shown in FIG. 7A, the primary implant body 10b can be positioned in an intervertebral space. The bone attachment material 11 can be pulled, pushed or folded back as shown in FIG. 7B. Then, as shown in FIG. 7C, the bone anchor 20b can be introduced into the target vertebral bone 25 proximate the implant 10. The bone can be “pre-drilled”, then the bone anchor inserted, or the bone anchor can be inserted without requiring pre-drilling. In other embodiments, the bone anchor(s) 20b can be introduced before the implant 10 and/or material 11. In still other embodiments, the bone attachment material 11 can be attached to the implant after the implant is in the body and/or after the bone anchor(s) is in position. As shown in FIG. 7D, in an exploded view for clarity, a suture set 22s can be pulled through the material 11. That is, the needles 23 can be inserted from one side of the material (i.e., flexible skirt) from the posterior (inner) to the anterior (outer) side. The suture set 22s can be pulled substantially taut and tied together to form a knot 22t against the outer surface of the material 11 while the bone anchor 20b remains in the vertebral bone 25 to tighten the material 11 against the vertebral body 25. The incision can then be closed with the knot 22t inside the incision (not pulled through the skin).

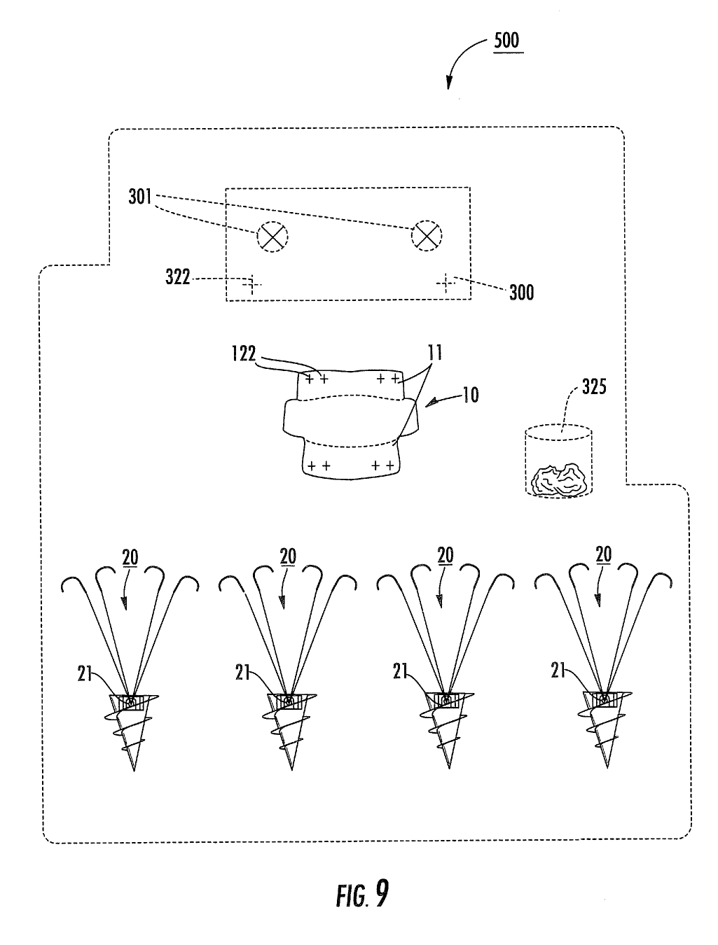

FIG. 8 illustrates three different exemplary mounting configurations for a suture anchor 20 that may be used to attach to spinal implants 10. As shown, two TDR implants 10 are in position in respective intevertebral spaces. The upper implant 10₁ includes a single level multi-attachment point suture anchor 20sm. The lower portion of the upper implant 10₁ and the upper portion of the lower implant 10₂ illustrate a double level multi-attachment point suture anchor 10dm. That is, sutures 22 from respective bone anchors 20b extend to different levels (above and below the bone anchors 20b). The lower level of the second implant 10₂ illustrates a single level, single attachment point suture anchor 20ss.

FIG. 9 illustrates a medical kit 500 that can provide the suture anchors 20. The kit 500 can include at least one implant 10 and a plurality of suture anchors 20. The kit 500 can also include the void filler 325 and at least one surgical template 300. The template 300 can include indicia for the bone anchor entry location 301 and may optionally include needle indicia 322 that can align with indicia on an interior surface of the bone attachment material 11 proximate the indicia 122 that can be placed on the outside surface of the material 11 (for indicating a target needle exit location). The template 300 may be configured so that each target bone anchor 20b location 301 is color-coded to bone anchors 20b and/or suture sets 22s and a location on material 11. A similar or different template 300 can be provided for attachment to a lower location or an upper location, or a combination template can be provided with both sets of alignment/target location indicia (not shown).

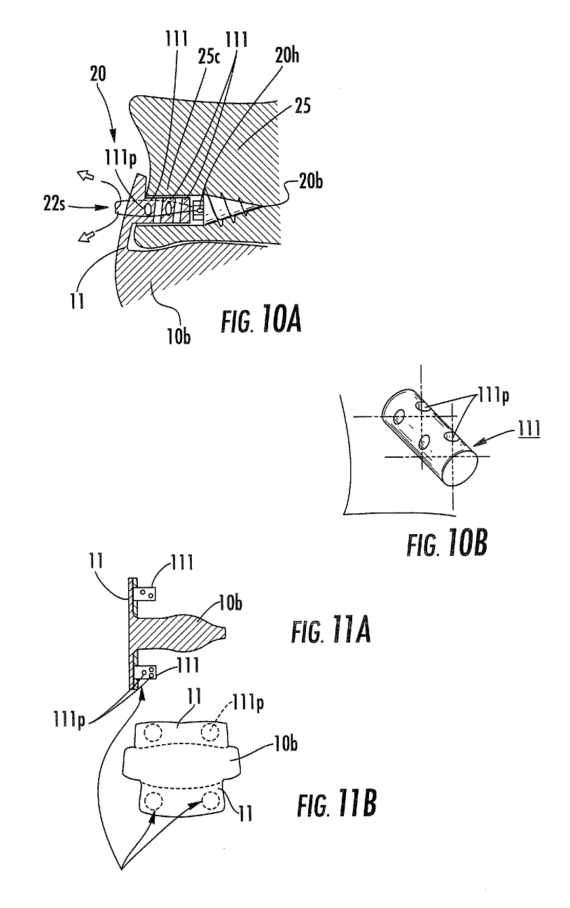

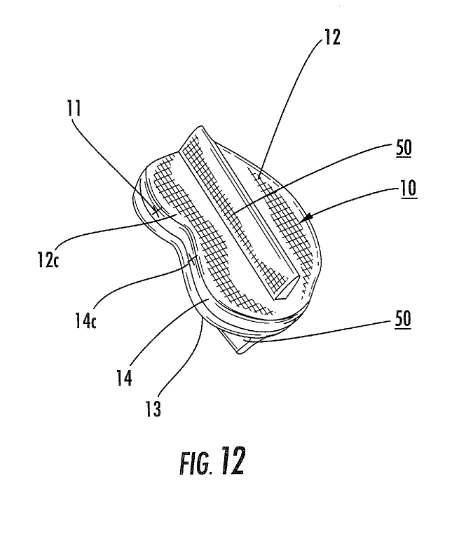

FIG. 10A illustrates that the bone anchor 20b can reside in a cavity 25c. FIGS. 10A, 11A and 11B illustrate that the attachment material 11 can include at least one plug 111 that is sized and shaped to enter the cavity 25c and reside between the bone anchor 20b and the outer perimeter of the bone and/or outer surface of material 11. The plug 111 can be attached to the attachment material 11 or be a separate component. FIG. 10B illustrates one exemplary shape of the plug 111. The plug 111 can comprise a metal, polymer or other suitable material. In some embodiments, the plug 111 is a mesh plug. The mesh plug 111 may comprise polyester fibers, such as DACRON and/or a polyvinylalcohol (PVA) cryogel. As shown in FIGS. 10A, 10B and 11A, the plug 111 can include macropores 111p. The plug 111 is typically a single one plug that has through holes 111p for bone to grow into. The bone growing in those through holes 111p can provide a solid long-term fixation of the plug 111 to the bone. The plug 111 can be integrally attached to the material 111 and/or the implant body 10. In some embodiments, the plug 111 is integrally attached to the skirt or tab material 11 and each may comprise a mesh fabric that is molded to the implant body 10b. FIG. 11A illustrates that the plug 111 faces into the bone and FIG. 11B illustrates the plug 111 can extend inward from a rear primary surface of the external attachment member (e.g., skirt or tab and the like). FIG. 10A illustrates that the bone anchor resides furthermost in the bone cavity with the plug(s) 111 residing between the external bone attachment member and the bone anchor 20b.

Referring to FIG. 12, in some embodiments, the shape of the implant 10 can be described as a three-dimensional structure that provides a desired anatomical shape, shock absorbency and mechanical support. In some embodiments, the anatomical shape can have an irregular solid volume to fill a target intervertebral disc space. The coordinates of the body can be described using the anatomic directions of superior (toward the head), inferior (toward the feet), lateral (away from the midline), medial (toward the midline), posterior (toward the back), and anterior (toward the front). From a superior view, the implanted device has a kidney shape with the hilum toward the posterior direction. The margins of the device in sagittal section are generally contained within the vertebral column dimensions. The term “primary surface” refers to one of the superior or inferior surfaces.

FIG. 12 illustrates one embodiment of spinal disc implant 10. The implant 10 can include at least one keel 50 on at least one primary surface. As shown, the implant 10 includes at least one flexible keel 50. In this embodiment, the flexible keel 15 is an anterior/posterior keel. In the embodiment shown in FIG. 12, the implant 10 includes both upper and lower keels 50 on respective superior and inferior primary surfaces. In other embodiments, the keel 50 can be oriented to extend substantially laterally. The keel 50 can be defined by a fold in a unitary layer of flexible material.

The size of the prosthetic spinal disc 10 can vary for different individuals. A typical size of an adult lumbar disc is 3-5 cm in the minor axis, 5 cm in the major axis, and 1.5 cm in thickness, but each of these dimensions can vary. It is contemplated that the implant 10 can be provided in a range of predetermined sizes to allow a clinician to choose an appropriate size for the patient. That is, the implant 10 can be provided in at least two different sizes with substantially the same shape. In some embodiments, the implant 10 can be provided in small, medium and large sizes. Further, the sizes can be configured according to the implant position—i.e., an L3-L4 implant may have a different size from an L4-L5 implant. In some embodiments, an implant 10 can be customized (sized) for each respective patient.

The implant 10 can be configured as a flexible elastomeric MRI and CT compatible implant of a shape generally similar to that of a spinal intervertebral disc. The implant 10 can have a solid elastomeric body with mechanical compressive and/or tensile elasticity that is typically less than about 100 MPa (and typically greater than 1 MPa), with an ultimate strength in tension generally greater than about 100 kPa, that can exhibit the flexibility to allow at least 2 degrees of rotation between the top and bottom faces with torsions greater than 0.01 N-m without failing. The implant 10 can be configured to withstand a compressive load greater than about 1 MPa.

The implant 10 can be made from any suitable elastomer capable of providing the desired shape, elasticity, biocompatibility, and strength parameters. The implant 10 can be configured with a single, uniform average durometer material and/or may have non-linear elasticity (i.e., it is not constant). The implant 10 may optionally be configured with a plurality of durometers, such as a dual durometer implant. The implant 10 can be configured to be stiffer in the middle, or stiffer on the outside perimeter. In some embodiments, the implant 10 can be configured to have a continuous stiffness change, instead of two distinct durometers. A lower durometer corresponds to a lower stiffness than the higher durometer area. For example, one region may have a compressive modulus that is between about 11-100 MPa, while the other region may have a compressive modulus that is between 1-10 MPa.

The implant 10 can have a tangent modulus of elasticity that is about 1-10 MPa, typically about 3-5 MPa, and a water content of between about 30-60%, typically about 50%.

Some embodiments of the implantable spinal disc 10 can comprise polyurethane, silicone, hydrogels, collagens, hyalurons, proteins and other synthetic polymers that are configured to have a desired range of elastomeric mechanical properties, such as a suitable compressive elastic stiffness and/or elastic modulus. Polymers such as silicone and polyurethane are generally known to have (compressive strength) elastic modulus values of less than 100 MPa. Hydrogels and collagens can also be made with compressive elasticity values less than 20 MPa and greater than 1.0 MPa. Silicone, polyurethane and some cryogels typically have an ultimate tensile strength greater than about 100 or 200 kiloPascals. Materials of this type can typically withstand torsions greater than 0.01 N-m without failing.

As shown in FIG. 12, the spinal disc body 10 may have a circumferential surface 11, a superior surface 12, and an inferior surface 13. The superior and inferior surfaces 11, 12 may be substantially convex to mate with concave vertebral bones. One or more of the surfaces may also be substantially planar or concave. The circumferential surface 11 of spinal disc body 10 corresponds to the annulus fibrosis (“annulus”) of the natural disc and can be described as the annulus surface 11. The superior surface 12 and the inferior surface 13 of spinal disc body 10 correspond to vertebral end plates (“end plates”) in the natural disc. The medial interior of spinal disc body 10 corresponds to the nucleus pulposus (“nucleus”) of the natural disc.

The implant 10 can include a porous covering, typically a mesh material layer, 12c, 13c on each of the superior and inferior primary surfaces 12, 13, respectively. As shown, the implant 10 can also include a porous, typically mesh, material layer 14c on the annulus surface 14. The annulus cover layer 14c can be formed as a continuous or seamed ring to inhibit lateral expansion. In other embodiments, the annulus cover layer 14c can be discontinuous. As also shown, the three coverings 12c, 13c, 14c can meet at respective edges thereof to encase the implant body 10. In other embodiments, the coverings 12c, 13c, 14c may not meet or may cover only a portion of their respective surfaces 12,13, 14.

FIG. 12 illustrates that the annulus cover 14c, the superior cover 12c, and or the inferior cover 13c can be oversized to extend beyond the bounds of the implant body 10b above or below an anterior portion of the implant body 10b to define the attachment material 11 that can cooperate with bone anchors 20b and sutures 22. The material 11 can extend above or below the body 10b with a height between about 2-35 mm, typically 5-15 mm.

The implant 10 may be configured to allow vertical passive expansion or growth of between about 1-40% in situ as the implant 10 absorbs or intakes liquid due to the presence of body fluids. The passive growth can be measured outside the body by placing an implant in saline at room temperature and pressure for 5-7 days, while held in a simulated spinal column in an intervertebrate space between two simulated vertebrates. It is noted that the passive expansion can vary depending, for example, on the type of covering or mesh employed and the implant material. For example, in some embodiments, the mesh coverings 14c, 12c, 13c along with a weight percentage of (PVA) used to form the implant body are configured to have between about 1-5% expansion in situ.

In addition, in some embodiments, the mesh may comprise a biocompatible coating or additional material on an outer and/or inner surface that can increase the stiffness. The stiffening coating or material can include PVA cryogel. The annulus cover 14C (also described as a “skirt”) can be a continuous skirt that defines the bone attachment material 11 and may include stiffening or reinforcement means.

Some embodiments of the spinal disc implant 10 are configured so that they can mechanically function as a substantially normal (natural) spinal disc and can attach to endplates of the adjacent vertebral bodies. As shown in FIG. 12, the spinal disc body 10b is generally of kidney shape when observed from the superior, or top, view, having an extended oval surface and an indented portion. The anterior portion of spinal disc 10 can have greater height than the posterior portion 10p of spinal disc 10 in the sagittal plane. The implant 10 can be configured with a mechanical compressive modulus of elasticity of about 1.0 MPa, ultimate stretch of greater than 15%, and ultimate strength of about 5 MPa. The device can support over 1200 N of force. Further description of an exemplary flexible implant is described in co-pending U.S. Patent Application Publication No. 20050055099, the contents of which are hereby incorporated by reference as if recited in full herein.

Elastomers useful in the practice of the invention include silicone rubber, polyurethane, polyvinyl alcohol (PVA) hydrogels, polyvinyl pyrrolidone, poly HEMA, HYPAN™ and Salubria® biomaterial. Methods for preparation of these polymers and copolymers are well known to the art. Examples of known processes for fabricating elastomeric cryogel material is described in U.S. Pat. Nos. 5,981,826 and 6,231,605, the contents of which are hereby incorporated by reference. See also, Peppas, Poly(vinyl alcohol)hydrogels prepared by freezing-thawing cyclic processing. Polymer, v. 33, pp. 3932-3936 (1992); Shauna R. Stauffer and Nikolaos A. Peppas.

In some embodiments, the implant body 10 is a substantially solid PVA hydrogel having a unitary body shaped to correspond to a natural spinal disc. An exemplary hydrogel suitable for forming a spinal implant is (highly) hydrolyzed crystalline poly(vinyl alcohol) (PVA). PVA cryogels may be prepared from commercially available PVA material, typically comprising powder, crystals or pellets, by any suitable methods known to those of skill in the art. Other materials may also be used, depending, for example, on the application and desired functionality. Additional reinforcing materials or coverings, radiopaque markers, calcium salt or other materials or components can be molded on and/or into the molded body. Alternatively, the implant can consist essentially of only the molded PVA body.

In some embodiments, the attachment material 11 is integrally attached to a moldable implant material via a molding process. The moldable primary implant material can be placed in a mold. The moldable material comprises an irrigant and/or solvent and about 20 to 70% (by weight) PVA powder crystals. The PVA powder crystals can have a MW of between about 124,000 to about 165,000, with about a 99.3-100% hydrolysis. The irrigant or solvent can be a solution of about 0.9% sodium chloride. The PVA crystals can be placed in the mold before the irrigant (no pre-mixing is required). The mold has the desired 3-D implant body shape. A lid can be used to close the mold. The closed mold can be evacuated or otherwise processed to remove air bubbles from the interior cavity. For example, the irrigant can be overfilled such that, when the lid is placed on (clamped or secured to) the mold, the excess liquid is forced out thereby removing air bubbles. In other embodiments, a vacuum can be in fluid communication with the mold cavity to lower the pressure in the chamber and remove the air bubbles. The PVA crystals and irrigant can be mixed once in the mold before and/or after the lid is closed. Alternatively, the mixing can occur naturally without active mechanical action during the heating process.

Typically, the mold with the moldable material is heated to a temperature of between about 80° C. to about 200° C. for a time sufficient to form a solid molded body. The temperature of the mold can be measured on an external surface. The mold can be heated to at least about 80-200° C. for at least about 5 minutes and less than about 8 hours, typically between about 10 minutes to about 4 hours. The (average or max and min) temperature can be measured in several external mold locations. The mold can also be placed in an oven and held in the oven for a desired time at a temperature sufficient to bring the mold and the moldable material to suitable temperatures. In some embodiments, the mold(s) can be held in an oven at about 100-200° C. for about 2-6 hours; the higher range may be used when several molds are placed therein, but different times and temperatures may be used depending on the heat source, such as the oven, the oven temperature, the configuration of the mold, and the number of items being heated.

The liners 14c, 12c, 13c can be placed in the mold to integrally attach to the molded implant body during the molding process. In some embodiments, osteoconductive material, such as, for example, calcium salt can be placed on the inner or outer surfaces of the covering layers 14c, 12c, 13c, and/or the inner mold surfaces (wall, ceiling, floor) to coat and/or impregnate the mesh material to provide osteoconductive, tissue-growth promoting coatings.

After heating, the implant body can be cooled passively or actively and/or frozen and thawed a plurality of times until a solid crystalline implant is formed with the desired mechanical properties. The molded implant body can be removed from the mold prior to the freezing and thawing or the freezing and thawing can be carried out with the implant in the mold. Alternatively, some of the freeze and thaw steps (such as, but not limited to, between about 0-10 cycles) can be carried out while the implant is in the mold, then others (such as, but not limited to, between about 5-20 cycles) can be carried out with the implant out of the mold.

Before, during and/or after freezing and thawing (but typically after demolding), the molded implant can be placed in water or saline (or both or, in some embodiments, neither). The device can be partially or completely dehydrated for implantation. The resulting prosthesis can have an elastic modulus of at least about 2 MPa and a mechanical ultimate strength in tension and compression of at least 1 MPa, preferably about 10 MPa, and under about 100 MPa. The prosthesis may allow for between about 1-10 degrees of rotation between the top and bottom faces with torsions of at least about 1 N-m without failing. The implant can be a single solid elastomeric material that is biocompatible by cytotoxicity and sensitivity testing specified by ISO (ISO 10993-5 1999: Biological evaluation of medical devices—Part 5: Tests for in vitro cytotoxicity and ISO 10993-10 2002: Biological Evaluation of medical devices—Part 10: Tests for irritation and delayed-type hypersensitivity).

The testing parameters used to evaluate the compressive tangential modulus of a material specimen can include:

Test type: unconfined compression

Fixtures: flat platens, at least 30 mm diameter

Rate: 25.4 mm/sec to 40% strain

Temperature: room temp (˜22° C.)

Bath: samples stored in saline or water until immediately before test

Samples: cylinders, 9.8±0.1 mm height, 9.05±0.03 mm diameter

Compressive Tangential Modulus calculated at 15, 20, and 35% strain

Embodiments of the instant invention employ anchors 20 to attach any suitable prosthesis and the present invention is not limited to spinal implants. In some embodiments, the suture anchors can be used to attach or affix implants comprising PVA cryogel material. The PVA cryogel implants can be manufactured to be mechanically strong, or to possess various levels of strength among other physical properties with a high water content, which provides desirable properties in numerous applications. For example, the cryogel tissue replacement construct is especially useful in surgical and other medical applications as an artificial material for replacing and reconstructing soft tissues or as orthopedic implants in humans and other mammals.

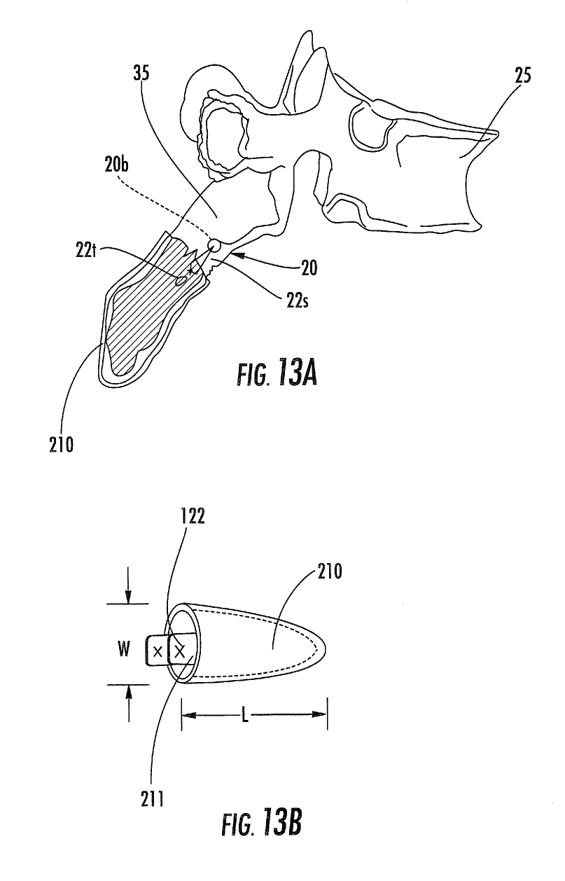

FIGS. 13A and 13B illustrate that suture anchors 20 can be used to secure other implants in the body. As shown in FIG. 13A, a spinous process sleeve or cuff implant 210 is in position on the spinous process 35 in the body. The suture anchor 20 is attached to the implant 210. That is, the bone anchor 20b resides in the spinous process 35 while the suture set 22sis tied 22tto the implant 210. FIG. 13B illustrates that attachment extensions 211 (such as tabs or a skirt) can be used to secure the sutures 22. The extensions 211 can include the needle indicia 122. FIG. 13A illustrates that the sutures 22 may be attached directly to the cuff body. The cuff body may include reinforced regions (i.e., PVA cryogel with polymeric mesh fabric, laminated layers of mesh fabric and the like) with increased rigidity or strength that inhibits tearing that define the attachment zones.

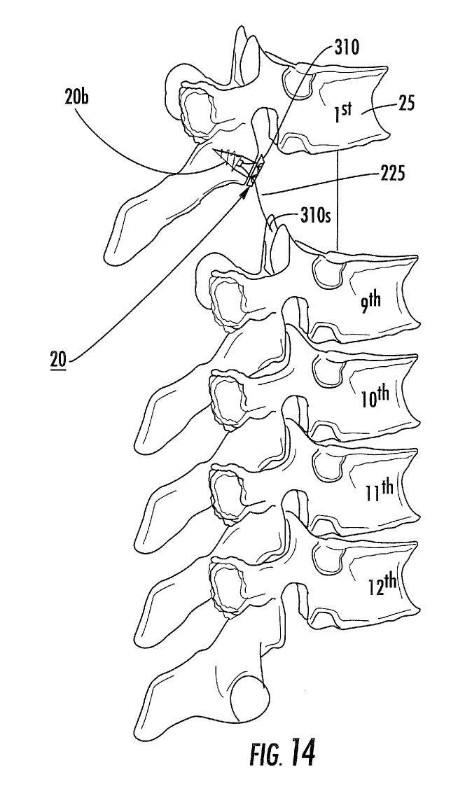

FIG. 14 illustrates a synthetic wide range facet implant 310 secured in position in the spine using a cooperating suture anchor 20. The implant 310 is configured as a “spinal facet joint” or joint surface. This term refers to the location at which vertebral bodies meet at a rear portion of the spine. The shape of facet joints change along the length of the spine. The facet joint includes bone, cartilage, synovial tissue, and menisci. The implant 310 can be an elastic body that is configured to substantially conformably reside on an outer surface of the bone in a manner that allows a relatively wide range of motion between the bones forming the joint. Also, as shown in FIG. 14, the suture knots can be recessed within the implant 310 device (such as in a small cylindrical recess or well for example) so that the knots are inhibited from rubbing against the opposite articulating surface of the facet joint.

The implants 310 and 210 can be substantially “conformal” so as to have sufficient flexibility to substantially conform to a target structure's shape. The facet implant or prosthesis can be applied to one surface (one side) of the facet joint (the bone is resurfaced by the implant) or to both surfaces of the joint, and/or may reside therebetween as a spacer to compress in response to loads introduced by the cooperating bones at the facet joint and still allow motion therebetween. The implant may be an elastic body that is configured to conformably reside on an outer surface of the bone in a manner that allows a relatively wide range of motion between the bones forming the joint. A facet implant or prosthesis can be applied to one surface (one side) of the facet joint (the bone is resurfaced by the implant) or to both surfaces of the joint, and/or may reside therebetween as a spacer to compress in response to loads introduced by the cooperating bones at the facet joint and still allow motion therebetween.

The spinal facet joint implant 310 can be configured to provide “wide range motion”; this phrase refers to the substantially natural motion of the bones in the facet joint which typically include all ranges of motion (torsion, lateral and vertical). The term “wide range motion” refers to substantially natural motion of the bones in the facet joint, which typically include the three motions associated with a functional spine unit, flexion/extension, lateral bending, and axial rotation. The motions translate differently in the disc compared to the facets but these motions are a good reference as far as range of motion. A facet joint sees sliding motions (along the joint surface) as well as compression and tension (in which case the facets are not in contact and the load is taken by the ligament only (capsular ligament)). The term “compact” means that the device is small with a low profile and suitable for surgical introduction into the spine. The term “thin” means that the device has a thickness that is less than about 6 mm, typically between about 0.001-3 mm, and may be between about 0.01 mm to about 0.5 mm. The term “conformal” means that the implant material or member is sufficiently flexible to conform to a target structure's shape. The target structure's shape can be either the upper portion of the lower bone or the lower portion of the upper bone (one of the two vertebral bones) that meet at the rear of the spine or both.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

That which is claimed:

1. A spinal implant with cooperating suture anchors, comprising:
a spinal implant; and
at least one suture anchor comprising a threaded bone anchor holding at least one suture, wherein, in position, the at least one suture extends outwardly from the threaded bone anchor and attaches to the spinal implant while the threaded bone anchor is anchored in a vertebral body.

2. An implant according to claim 1, wherein the spinal implant is a total disc replacement spinal implant with bone attachment material attached thereto, the spinal implant having a primary implant body with a boundary substantially coextensive with a target natural spinal disc replacement location, wherein the bone attachment material extends in at least one of a superior or inferior direction beyond the bounds of the primary implant body with the at least one suture extending through the bone attachment material while attached to the threaded bone anchor in the vertebral body.

3. An implant according to claim 1, wherein the suture includes at least one needle, wherein the needle is curved with a substantially blunt tip.

4. An implant according to claim 1, wherein the spinal implant comprises bone attachment material, wherein the at least one suture is configured as a suture set so that a length of suture forms two outwardly extending legs with a medial portion therebetween, with each of the two legs having a needle on an end portion thereof, and wherein, when attached to the bone attachment material, the two legs of the suture define a suture knot that reside tightly against an outer side of the bone attachment material with the threaded bone anchor residing on the other side of the bone attachment material recessed into the vertebral body.

5. An implant according to claim 4, wherein the at least one threaded bone anchor is a plurality of threaded bone anchors each comprising at least one suture set for multi-point attachment to the bone attachment material.

6. An implant according to claim 1, wherein the at least one threaded bone anchor includes at least one bone anchor that is attached to a plurality of suture sets to provide a multi-point attachment to the bone attachment material.

7. An implant according to claim 2, wherein the threaded anchor comprises a body with a head and a suture attachment region, and wherein the suture attachment region is recessed a distance into the anchor body.

8. An implant according to claim 7, wherein at least a portion of the threaded body is sized and configured to attach to vertebral cancellous bone, and wherein the head of the anchor is adapted to be flush with or recessed into the vertebral body.

9. An implant according to claim 1, wherein the anchor has a diameter of between about 5-8 mm and a length between about 10-20 mm.

10. An implant according to claim 1, wherein the at least one threaded anchor is resorbable.

11. An implant according to claim 1, wherein the suture is resorbable.

12. An implant according to claim 2, wherein the primary body is a non-articulating unitary elastomeric body.

13. An implant according to claim 2, wherein the bone attachment material comprises a porous fabric.

14. An implant according to claim 13, wherein the bone attachment material comprises a mesh skirt extending over at least a major portion of an annulus outer surface of the primary implant body with upwardly and downwardly extending pliant segments, and wherein the at least one threaded bone anchor comprises four spaced apart threaded bone anchors with suture sets that are secured to the mesh skirt via tied suture knots from respective sutures sets, the tied suture knots snugly abut an outer surface of the pliant segments forcing the bone attachment material against the vertebral body.

15. An implant according to claim 1, wherein the implant comprises bone attachment material with at least one inwardly extending mesh plug with macropores that resides in a respective canal formed in the vertebral body between a respective threaded bone anchor and an inner surface of the bone attachment material, wherein, in position the at least one mesh plug is substantially axially aligned with the threaded anchor.

16. An implant according to claim 2, wherein the primary body comprises a crystalline polyvinylalcohol hydrogel, wherein the bone attachment material comprises a mesh skirt with attachment segments that extend above and below a superior and inferior surface of the primary body, and wherein the at least one threaded bone anchor is at least four threaded bone anchors, a respective one attached to spaced apart portions of the attachment segments of the mesh skirt with the suture sets from the bone anchors tied in knots tightly against an outer surface of the mesh skirt.

17. An implant according to claim 1, wherein the at least one threaded bone anchor includes a plurality of threaded bone anchors that have a plurality of suture sets with at least two suture sets residing in a first threaded bone anchor and extending from a first vertebral body above the implant and at least two suture sets residing in a second threaded bone anchor residing in a second vertebral body below the implant, whereby a respective suture pair resides above and below the implant body.

18. A medical spinal implant kit, comprising;
a total disc replacement (TDR) spinal implant comprising a bone attachment material; and
a plurality of suture anchors configured to define suture knots against an outer surface of the bone attachment material with the threaded anchors configured and sized to reside in at least one vertebral body above or below the TDR implant to secure the TDR implant in position.

19. A medical kit according to claim 18, further comprising a sterile package enclosing the TDR and suture anchors.

20. A medical kit according to claim 18, wherein the bone attachment material comprises target tie location indicia to allow a clinician to align suture knots at desired locations on the bone attachment material.

21. A medical kit according to claim 18, wherein the kit further comprises bone filler material.

22. A medical kit according to claim 18, wherein the suture anchors comprise a threaded bone anchor body and a plurality of suture sets.

23. A medical kit according to claim 18, wherein the bone attachment material comprises mesh.

24. A method of attaching a total disc replacement (TDR) implant to at least one vertebral body, comprising;
implanting a TDR;
anchoring at least one bone anchor in at least one vertebral body proximate the TDR; and
tying at least one suture set attached to the bone anchor to the TDR to thereby secure the TDR in position in the body.

25. A method according to claim 24, wherein the anchoring step is carried out after the implanting step.

26. A method according to claim 24, wherein the implanting step is carried out before the anchoring step.

27. A method according to claim 24, wherein the TDR comprises a porous bone attachment material extending upwardly and/or downwardly beyond the bounds of the primary implant body, wherein the tying step comprises:
pushing the porous bone attachment material away from the proximate vertebral body during the anchoring step; and pulling needles from the suture set through the porous bone attachment material before the tying step.

28. A method according to claim 24, wherein the TDR implant comprises an elastomeric primary body with mesh bone attachment material integrally attached thereto, wherein the anchoring step comprises anchoring a plurality of spaced apart threaded bone anchors in the at least one vertebral body, each threaded bone anchor comprising at least one suture set, wherein the tying step comprises tying a plurality of spaced apart suture sets tightly against the mesh material.

29. A method according to claim 24, wherein the anchoring step comprises anchoring the bone anchor in the vertebral body so that a tip portion thereof engages cancellous bone and an opposing head portion thereof is flush or recessed into the vertebral body.

30. A method according to claim 29, wherein the head portion resides inside the cortical layer.

31. A method according to claim 28, wherein the mesh material comprises a mesh plug that is configured to reside in a bone canal in communication with the bone anchor to thereby promote bone growth.

32. A method according to claim 24, wherein the anchoring at least one bone anchor in at least one vertebral body proximate the TDR comprises anchoring two spaced apart bone anchors in an upper vertebral body and anchoring two spaced apart bone anchors in a lower vertebral body with the TDR therebetween, and wherein the tying step comprises tying at least four suture sets, at least one set attached to a respective anchored bone anchor to the TDR to thereby secure the TDR in position in the body.

33. A TDR implant, comprising:
a flexible implant body; and
a bone attachment member with at least one outwardly extending plug configured and sized to reside in a cavity formed in a vertebral body.

34. A TDR implant according to claim 33, further comprising at least one threaded bone anchor with at least one suture set attached to the bone attachment member, wherein a single bone anchor is sized and configured to reside in the vertebral cavity with a respective plug.
